(19) United States
(12) Patent Application Publication
(10) Pub. No.: US 2008/0045807 A1
(43) Pub. Date: Feb. 21, 2008

(54) System and methods for evaluating and monitoring wounds

(76) Inventors: Eric T. Psota, Lincoln, NE (US); Lance C. Perez, Lincoln, NE (US)

Correspondence Address: VALAUSKAS & PINE LLC, Suite 1825, 150 North Wacker Drive, Chicago, IL, 60606, US

(21) Appl. No.: 11/811,051

(22) Filed: Jun. 8, 2007

Related U.S. Application Data
(60) Provisional application No. 60/812,575, filed on Jun. 9, 2006.

(51) Int. Cl. A61B 5/00

(52) U.S. Cl. 600/300; 606/130

(57) ABSTRACT

A system and methods to evaluate and monitor the healing progress of a wound. At least two optical imaging devices are mounted on a support device in order to capture images. The images are resolved in order to be matched to create a depth map of the object. The depth map can be analyzed to obtain data, such as the length, width, and depth of the wound and the area and volume of the wound.

This application claims the benefit of U.S. Provisional Application No. 60/812,575 filed Jun. 9, 2006.

### FIELD OF THE INVENTION

The present invention relates generally to wounds. More particularly, the present invention relates to obtaining data of wounds to evaluate and monitor the progress of healing.

### BACKGROUND OF THE INVENTION

There are various types of wounds. A wound is a type of physical trauma wherein the skin is torn, cut or punctured. Some occur in the first few layers of skin, while others occur in deeper layers of skin. Examples of wounds include incisions, lacerations, ulcers, abrasions, puncture, penetration, and gunshot to name a few. Wounds are defined by different nomenclature, which is related to the diagnosis, such that different wounds are treated differently.

Typically, an initial evaluation of each wound is made along with a continuous evaluation, or monitoring, to assess healing. Evaluation and monitoring of the wound gives a treating medical practitioner a history and progress of wound healing, which in turn is a direct reflection of a chosen treatment regimen.

Wounds are three dimensional and, as such, are difficult to measure. All measurement methods or techniques have to deal with three general problems that directly affect accuracy: definition of wound boundary, changing shape of wounds, and the natural curvature of the body's surface.

Defining the boundary of a wound is often difficult. It always depends on the subjective judgment of the human observer who performs the measurements to define whether a particular part of the area in question belongs to the wound or not.

Wounds change shape because they are largely flexible. Slight movements, the flexing of a muscle, or a change in the patient's position, may significantly change the appearance of a wound. In such cases, the reproducibility of measurements is often more a function of the patient's position rather than the accuracy of the respective measurement process itself.

Another general problem is the natural curvature of the human body. Wounds may extend well around a limb which poses problems to techniques based on photography or other optical methods. Even if the wound is fully visible to the measurement device, those methods which ignore surface curvature will produce inaccurate results.

Deep wounds have a tendency to heal first from their base rather than their perimeter. Therefore a variety of attempts have been made to assess the volume of wounds. In addition to the problems of boundary definition, changing wound shape, and body curvature, some wounds are also extensively undermined and may change their volume with the patient's position.

In spite of the above problems, a variety of attempts have been made to measure the area and volume of wounds. A number of methods for measuring area of wounds exist including these using ruler-like measuring techniques, transparency tracing methods, and optical methods. The practice of producing casts or filling the wound with saline are typically used to determine the volume of a wound.

Accurate measurements of the physical size, such as area and volume, of a wound are vital for assessment of the progress of healing. One such method to evaluate and monitor a wound utilizes a ruler that is placed over a wound and by which its length and width is determinable. This same ruler is then placed vertically into a wound and by which a second depth measurement may be taken. Disadvantages of ruler-based methods include a high standard of deviation between measurements, inaccuracy—, since measurements are not always taken under identical conditions, or by the same personnel—, and contact with the wound by the ruler.

Transparency tracing methods typically use a sterilized sheet of transparent acetate placed on the wound. The wound's perimeter is then traced using a permanent marker pen. Alternatively, sterile transparency material, that is flexible and follows skin curvature, is used with an imprinted metric grid. The main source of error for this method is the ability of observers to define the edge of a wound precisely. Other disadvantages associated with this method arise from the contact that must be made with the wound and that the method is time consuming (since area is calculated typically by counting grids or boxes on the paper).

While optical methods, which include photographic techniques and stereo-optical techniques, avoid direct contact with the wound, the accuracy of these methods is reduced by the need to scale the photographs and by the curvature of wound area. These methods may not be used if the wound stretches around a curved surface, or limb, and is not entirely visible to the camera. Optical methods also have poor repeatability and the equipment needed to carry out the methods is expensive. Additionally, some optical methods, such as stereo-optical techniques require special training.

The use of casts to determine the volume involves typically filling a wound with various substances such as molding material. After the wound is filled with the molding material and is hardened, the molding material is then removed from a wound site and measured. The disadvantages of the molding method are that it is painful to a patient and disregards good sterile technique. A less painful, but less accurate method to determine volume involves filling a wound with fluid, such as normal saline and recording the volume of fluid used. This technique is disadvantageous in that the patient must remain absolutely still to avoid the loss of any fluid during the filling process.

Overall, all known techniques are largely disadvantageous in that they are manual systems and methods are invasive and lack precise and consistent data.

There is therefore a demand for a non-invasive procedure to evaluate and monitor wounds, while obtaining precise and consistent data, such as measurements of wound features, to assess the progress of healing. The present invention satisfies this demand.

### SUMMARY OF THE INVENTION

For purposes of this application, the present invention is discussed in reference to wounds, but the present invention is applicable to any object for which data can be conveyed using stereoscopy, for example, aerial data of large geographic areas.

Stereoscopy, or stereoscopic imaging, or 3-D (three-dimensional) imaging is any technique capable of creating the illusion of depth in a 2-D (two-dimensional) image, such as a photograph, movie, display, drawing, painting, carving, computer representation. The illusion of depth in a 2-D image is created by presenting a slightly different image to each eye.

The present invention is a non-invasive procedure to evaluate and monitor wounds, while providing precise and consistent data of the wound features to assess the progress of healing and the efficacy of a given course of medical treatment. A wound for purposes of this application is an area of the body of a person or animal involving at least the skin that is damaged unintentionally—such as because of an accident—or which is cut, punctured, or treated—such as because of a medical operation. A wound for purposes of this application can include skin blemishes or discolorations, for example, and the invention may be used to detect or monitor possible melanoma or other skin conditions. Data includes measurements of area and volume, although it is contemplated that the present invention can obtain data including color, odor, temperature, and condition of the tissue. Area data includes measurements of size, such a length, width, and depth. Volume data includes measurements of the amount of space occupied by the 3-D wound.

The present invention is a stereoscopic apparatus that includes an optical imaging device, support device, and a computational device. It may include a projection device.

The optical imaging device is one or more devices that captures at least two 2-D images, for example, a first image and a second image. The optical imaging device can be a camera, stereo camera, digital camera, and a computer-based web camera.

The support device provides stability and possibly mobility to the stereoscopic apparatus of the present invention. The support device can, for example, be a mounting stand. In one embodiment, the stereoscopic apparatus includes a mounting stand such that the stand preferably is capable of extending over raised surfaces, such as a bed. The ability for the stand to boom, or telescope, is preferred in order that the optical imaging device may be positionable generally adjacent to the object being evaluated and monitored, for example, a wound on a patient in a hospital bed, while at the same time keeping the optical imaging device steady for optimum image quality. In instances where the object of interest was able to be moved easily—for example, if the patient was able to stand and maneuver the wound in front of the optical imaging device—the booming capability may not be necessary. In these instances, the support device can be a tri-pod type stand.

It is contemplated the support device be adjustable in the vertical direction that is, above and away from the wound. It is also contemplated that the support device be sufficiently flexible in order that wounds that are not on flat services—such as areas having contours—in order to be evaluated from various directions so as to capture images from multiple angles.

In addition, it is contemplated that embodiments in accordance with the present invention may include components by which the support device is made more easily movable thereby improving the mobility and utility of the stereoscopic apparatus. Wheels at the bottom of the support device are examples of such mobility components. In a hospital, for example, this would allow a nurse or aide to be able to conduct patient checks in multiple rooms efficiently. It is contemplated that the stereoscopic apparatus may also include the ability to store settings for the device for individual patients, such that a nurse would be able to store the image angles for an individual patient within the stereoscopic apparatus. This would assist in the ease of mobility of the device and also increase the reliability that consecutive images could be compared easily over time from the same image angles.

Preferably attached to the support device is an optical device mount, such as a bracket, and, in applications that may have reduced lighting, a lighting source. It is contemplated that the support device and the optical device mount may be integrated to form one piece or, alternatively, may be two separate pieces. Further, the optical device mount is adapted to be attached to a plurality of optical imaging devices. In embodiments with a plurality of optical imaging devices, certain embodiments of the invention include the devices placed equidistant relative to the center of the support stand, such that the object being evaluated and monitored is lined up with the support stand. Preferably, the optical device mount is made from a rigid material, including metal, such as aluminum. It is contemplated, however, that the optical device mount may also be made from wood or a plastic.

The optical device mount is also preferably rotatably adjustable, such that, for example, equidistant optical imaging devices mounted on the support device can be rotated toward each other or away from each other. Such a feature facilitates the calibration of the optical imaging devices such as horizontal alignment. It is also contemplated that the optical device mount is adjustable horizontally which in certain embodiments is perpendicular to the support device. This allows for the optical imaging devices to be placed as far apart or as close together as necessary to achieve the desired image. The exact position may depend on the size of the object of interest. Thus, the distance between the optical imaging devices varies depending on the relative size of the object to be imaged. As one example, when monitoring a wound approximately four square centimeters in total area, certain embodiments of the apparatus include the placement of the optical imaging devices with generally little distance between them due to the relative small size of the object of interest. Preferably, the optical imaging devices capture the same object in their respective fields of view, such that the images overlap, and the object captured utilizes as many pixels as possible within the images.

As mentioned above, in accordance with the present invention, the optical imaging devices may be two digital cameras. As an example, the digital cameras are mounted to the optical device mount using standard tri-pod mounting screw holes in the bottom of the cameras. In order to make sure that cameras are able to take pictures simultaneously, it is preferred that a single button be hard-wired into each of the cameras so that by the depression of the single button the optical imaging devices may be actuated to capture an image from both cameras. In one embodiment, the single button simply shorts the button on top of the cameras that are normally used to capture an image, or picture.

The present invention may optionally include a projector device, such as a laser, or other deterministic optical reference. The laser projects a reference chart, such as a graph, diagram or grid. A laser grid projector is adapted to be utilized in stereoscopic apparatuses. Since not all objects, like wounds, have defined contours, it may become necessary to utilize a laser projecting a reference chart on the object. For example, the use of a laser grid is preferred in instances where the wound is large and/or when the wounded area has very little structure for matching. A laser grid is a laser projecting a grid on the object. Laser grids are projected onto the object to be imaged and provide easily recognizable contours to the images to be captured in order to match, or overlap, a plurality of contours upon the image being captured. It is contemplated, however, that, where an object for imaging has easily matched contours, the laser grid may not be necessary.

Preferably, the projector device is mounted to the optical device mount with long, flexible tubing, such as metal tubing. This flexibility allows the user to adjust the position of the laser relative to the optical imaging devices similar to that described above in reference to the optical imaging device. It is also contemplated that the projector device may be integrated with the support stand.

Once the images have been captured by the optical imaging devices, the images may be provided, or downloaded, to a computational device. The optical imaging devices can provide the images to the computational device in any number of ways, as would be appreciated by those of ordinary skill in the art. For example, the optical imaging devices may include a cable that is connected to the computational device. Alternatively, the optical imaging devices may include a wireless connection to the computational device, such as Bluetooth® technology, in order to provide images to the computational device.

It is also contemplated that the computational device, for use in embodiments of the present invention, includes a computer program in order to resolve the images that have been provided by the optical imaging devices. The computer program for use with the present invention may include components by which the images from each of the optical imaging devices may be resolved in order to match the images. Resolving the optical images includes recognizing contours within each image such that one contour in a first image is matched to the exact contour in a second image, etc. This allows the images to be optimized for image quality. The image may then be analyzed in order to obtain data on the object, such as a wound, non-invasively. Data can include area and volume of the object, including length, width, and depth.

It is contemplated that the program of the computational device may include setting the time intervals for capturing optical images and therefore may include the ability to actuate the optical imaging devices. The computational device may include a display and a processor, such as a computer. The processor executes the program that allows a plurality of images to be resolved and matched to create a depth map for the object. The depth map illustrates various contours of the object. The processor may further store the depth map to allow a user to analyze the wound, such as obtaining data. For example, the user can select points of interest within the image. The points of interest may be used in order to measure area and volume of the wound, including length, width, and depth. The program may allow for analysis of the wound as would be appreciated by those of ordinary skill, such as an analysis of the wound's progress of healing.

An object of the present invention is to provide a quick evaluation of the healing of wounds in humans and animals.

Another object of the present invention is to provide precise and accurate measurements of a wound or wounds.

An added object of the present invention is to measure wound features or contours to obtain data that can be analyzed to determine the progress of a wound's healing.

An additional object of the present invention is to capture contours of an image which may not necessarily be visible to the naked eye.

A further object of the present invention is to enable images of wounds to be obtained through the use of simplified methods and apparatus even by unskilled individuals.

### BRIEF DESCRIPTION OF THE DRAWINGS

The patent or application contains at least one drawing executed in color. Copies of this patent or application with color drawings will be provided by the Patent Office upon request and payment of the necessary fee.

FIG. 1 illustrates an embodiment according to the present invention;

FIG. 2 illustrates a flow chart of a computer program according to the present invention;

FIG. 3 illustrates a captured first image and a captured second image according to the present invention; and

FIG. 4 illustrates a depth map according to the present invention.

### DETAILED DESCRIPTION OF AN EMBODIMENT OF THE INVENTION

In accordance with one embodiment of the present invention, a system and methods for monitoring the healing of wounds is provided. Although described in detail with respect to one embodiment of the present invention, it is to be appreciated that the teachings of the present invention are amenable to other applications, such as, but not limited to any application for which data is sought using stereoscopic imaging, including the collection of data of large geographic areas using aerial images. The present invention is also useful to collect images over a period of time and facilitate the objective comparison of the same. The images that result from the use of the present invention can be taken and subjected to contemporaneous analyses in one place or sent to another location for generally contemporaneous or subsequent analyses and then returned to the site of origination. In this way, images of wounds may be taken on battle fields, field hospitals, regional health clinics, and sent to other locations for storage, analysis, and reporting.

For convenience of description, terms such as “upper”, “lower”, “outer”, “inner”, “horizontal”, “vertical” “outwardly”, and “inwardly” are used to refer to the apparatus 100 and the components of inventory associated with the apparatus 100 in an orientation illustrated in the accompanying drawings. However, it will be understood that the embodiments of the invention described in this application advantageously can be used in a variety of orientations.

In accordance with one embodiment of the present invention, a stereoscopic apparatus is utilized for monitoring wounds. The stereoscopic apparatus preferably includes two optical imaging devices, such as digital cameras, that capture the image for each of the cameras field of view. The images are inputed into a computer program that allows the images to be matched to each other to obtain a depth map of the wound. The user may then select points of interest around and inside of the wound.

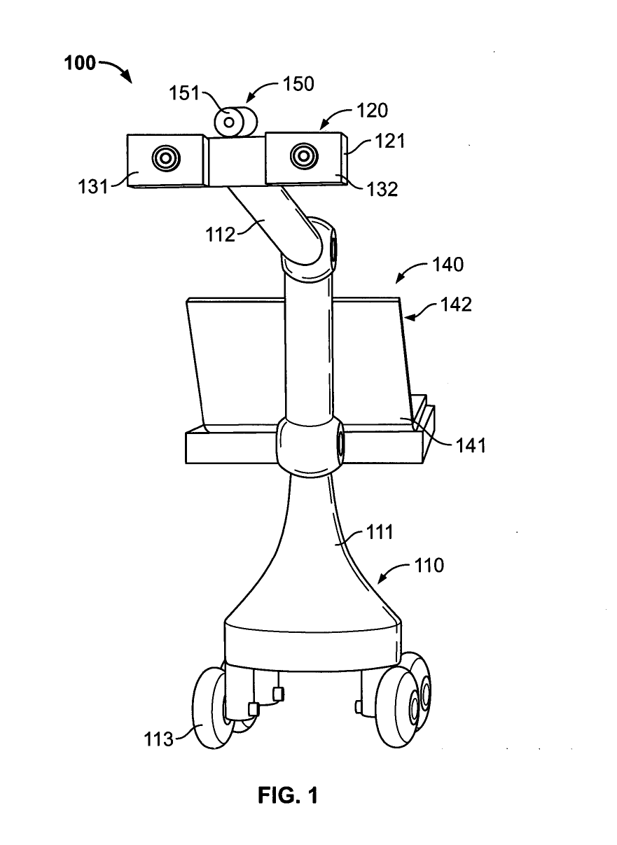

Illustrated in FIG. 1 is an embodiment in accordance with the present invention is shown. The illustrated the stereoscopic apparatus 100 includes a support device 110, an optical device mount 120, an optical imaging device 130, a computational device 140, and a projection device 150. As shown, the support device 110 is a mounting stand 111 that includes a booming portion 112 and an optical device mount 120, the illustrated embodiment includes a bracket 121 as the mount 120. Furthermore, in this embodiment, the optical imaging device 130 is adapted to be integrated within the optical device mount 120. Also, in this embodiment, the computational device 140 is adapted to be fixed to the support device 110 such that moving the stereoscopic apparatus 100 will also move the computational device 140. In order to improve mobility of the stereoscopic apparatus 100, the illustrated embodiment includes one or more mobility components 113—such as the illustrated wheels—to the bottom of the support device 110.

The booming portion 112 of the mounting stand 111 allows the optical imaging device 130 mounted on the stereoscopic apparatus 100 to be adjusted so that the device 130 is positionable in closer proximity to the wound without having to move the entire apparatus 100. As illustrated, the support device 110 includes various levels of flexibility and adjustability. For example, the embodiment illustration FIG. 1 includes a support device 110 that is rotatable around its vertical axis, adjustable vertically, and can be shortened or lengthened because of the booming portion 112 of the mounting stand 111. Allowing the support device 110 to be adjustable in many directions facilitates proximity to the wound, which aids in monitoring and analyzing the wound.

As illustrated in FIG. 1, the optical device mount 120 is preferably attached to the support device 110 in a perpendicular fashion so as to allow an optical imaging device 130 to be mounted to the optical device mount 120. Preferably, the optical device mount 120 is a rigid piece that may be made of metal, wood or polymer. It is also contemplated that embodiments in accordance with the present invention utilize a horizontally adjustable optical device mount 120 so as to allow the optical imaging device 130, once mounted, to be adjustable so as to place the optical imaging device 130 as far apart or as close together with another optical imaging device 130, as necessary.

The embodiment shown in FIG. 1 includes a bracket 121 integrated with the mounting stand 111. Attached to the bracket 121, are preferably two optical imaging devices 130, shown in the illustrated embodiment as digital cameras 131, 132. The digital cameras 131, 132 provide images to a computational device 140, such as a computer 141 that preferably includes a display 142.

The digital cameras 131, 132 are rotatably mounted onto the bracket 121. As such, the optical imaging device 130 can preferably be rotated inwards or outwards, depending on the desired view for the wound being monitored. As can also be seen, the digital cameras 131, 132 are preferably mounted on the mounting stand 111 to be equidistant from the center of the support device 110. Preferably, the wound that is being monitored is located to be equidistant from each of the digital cameras 131, 132, although any location arrangement is contemplated.

The embodiment shown in FIG. 1 includes a projector device 150. The projector device 150 is a laser grid projector 151. As explained above, the laser grid projector 151 “throws” a grid via a laser onto the wound being monitored. The laser grid projector 151 may be encased in flexible tubing thereby permitting the user to adjust the output of the laser grid projector, while maintaining its position while adjusted. It is contemplated that this can be achieved in any number of ways as would be appreciated by those of ordinary skill in the art. The laser grid projector 151 may also be integrated with the support device 110.

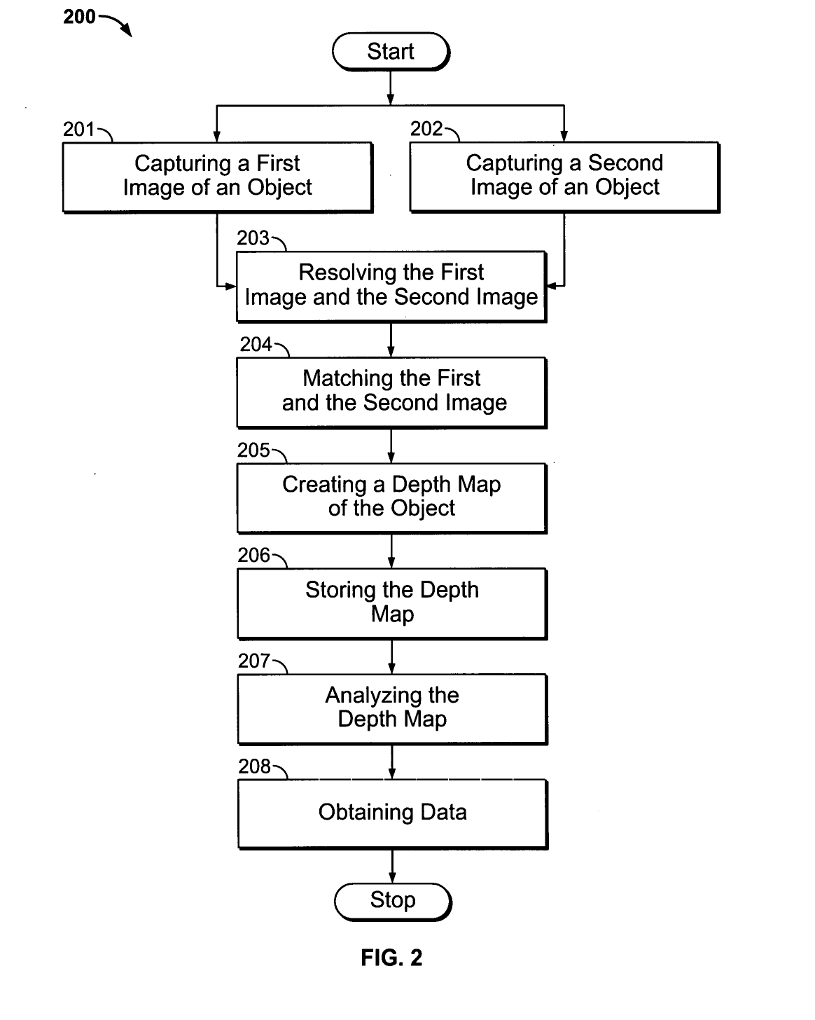

FIG. 2 illustrates a flow chart of a computer program 200 of a computation device according to the present invention. A first image and a second image of an object are captured as shown in steps 201 and 202, respectively. After the images are captured at steps 201 and 202, the images are resolved at step 203 by the recognition of the contours of each image such that the contours in each image are matched, or overlapped, at step 204. At step 205, a depth map is created. The depth map is stored within the computational device at step 206. The depth map is then analyzed at step 207 to obtain data, such as area and volume, including length, width, and depth, at step 208.

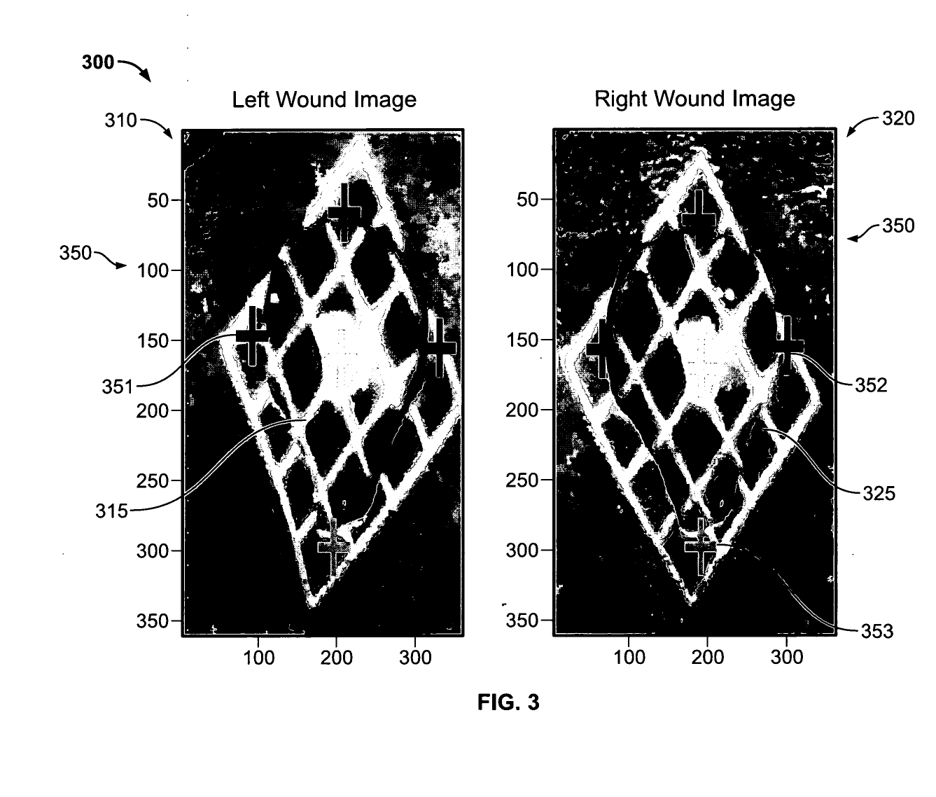

As illustrated in FIG. 3 optical imaging devices for use in accordance with the teachings of the present invention render images of the wound 300. As illustrated, the stereoscopic apparatus preferably captures two images of the wound 300, a left wound image 310 and a right wound image 320. Both images 310, 320 display the wound 300. In addition, FIG. 3 illustrates the utilization of a projector device in conjunction with a stereoscopic apparatus in accordance with the present invention. The projector device projects grids 315, 325 onto the wound, thereby allowing for the easier matching of contours on the wound between a left wound image 310 and the right wound image 320. In addition, by using a computer program, various points 350 can be selected, such as points 351 on the left wound image 310 and points 352, 353 on the right wound image 320.

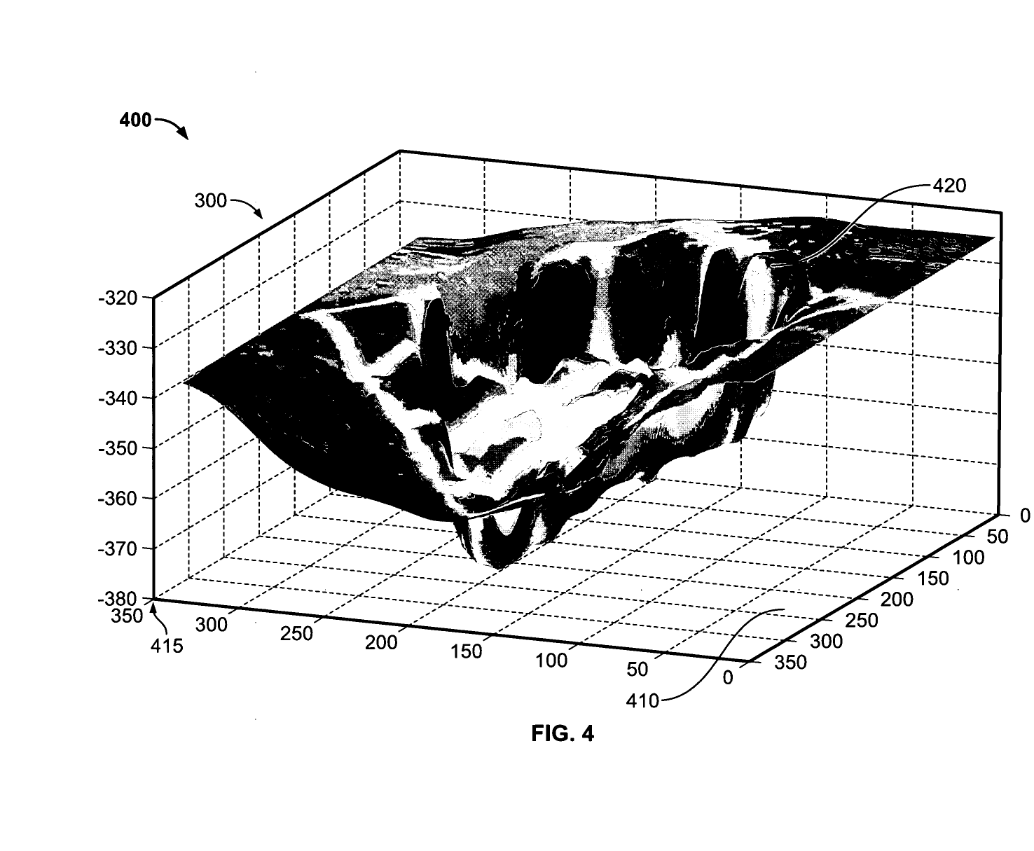

A computer created depth map 400 of a wound being evaluated and monitored in accordance with the teachings of the present invention is shown in FIG. 4. As shown, the wound 300 is depicted in a three-dimensional depth map 410. The depth map 410 shows the contours 420 of the wound, including area and volume of the wound, as well as length, width, and depth. In addition, grid 415 assists in locating features of the wound.

The invention has been described with reference to preferred embodiments. Obviously, modifications and alterations will occur to others upon a reading and understanding of this specification. It is intended that the invention be construed as including all such modifications and alterations insofar as they come within the scope of the appended claims or the equivalents thereof.

What is claimed is:

1. A method for evaluating and monitoring a wound, comprising the steps of:
positioning a stereoscopic apparatus within proximity of the wound;
actuating the stereoscopic apparatus;
capturing a first image and a second image of the wound;
resolving the first image and the second image;
matching the first image and the second image;
creating a depth map of the wound;
storing the depth map of the wound;
analyzing the depth map; and
obtaining data of the wound.

2. The method of claim 1, wherein said step of resolving said first image and said second image further includes recognizing contours of the wound from each of the first image and the second image.

3. The method of claim 1, wherein said step of obtaining data of the wound includes the data from at least one from the following group of: area of the wound;
volume of the wound; length of the wound; width of the wound; and depth of the wound.

4. A stereoscopic apparatus for evaluating and monitoring a wound comprising:
a support device having a center point;
a first optical imaging device to capture a first image;
a second optical imaging device to capture a second image;
wherein said first optical imaging device and second optical imaging device are each positioned equidistant from said center point of said support device; and
a computational device to match the first image with the second image to create a depth map of the wound.

5. The apparatus of claim 4, further comprising a projector device to project a reference chart onto the wound.

6. The apparatus of claim 4, further comprising an optical device mount for mounting said first optical imaging device and said second optical imaging device.

7. The apparatus of claim 4, wherein said first optical imaging device and said second optical imaging device is a digital camera.

8. The apparatus of claim 5, wherein said projector device is a laser.

9. The apparatus of claim 5, wherein said reference chart is a grid.
